(19) United States
(12) Patent Application Publication
(10) Pub. No.: US 2004/0063152 A1
(43) Pub. Date: Apr. 1, 2004

(54) Method for electrochemical analysis, corresponding configurations and the use thereof

(76) Inventors: Walter Gumbrecht, Herzogenaurach (DE); Konrad Mund, Uttenreuth (DE); Manfred Stanzel, Erlangen (DE)

Correspondence Address: STAAS & HALSEY LLP, SUITE 700, 1201 NEW YORK AVENUE, N.W., WASHINGTON, DC, 20005, US

(21) Appl. No.: 10/432,781

(22) PCT Filed: Nov. 26, 2001
(86) PCT No.: PCT/DE01/04438

(30) Foreign Application Priority Data
Nov. 24, 2000 (DE) 100 58 397.0

(51) Int. Cl. G01N 33/53

(52) U.S. Cl. 435/7.1; 205/777.5

(57) ABSTRACT

Redox (re)cycling is improved in terms of the measurement technique in such a way that the redox potential created by a redox pair is measured on a reference electrode in an electroless manner. A configuration adapted to the method contains an electrode system having at least three electrodes: one working electrode, one counter electrode and one reference electrode. The reference electrode is arranged in such a way that it is adjacent to at least partial areas of the two other electrodes, and preferably, at an equal distance from the partial areas. In terms of redox recycling, the electrode system is suitable, for example, for detecting enzyme-coupled identification reactions, but also for measuring an oxygen partial pressure or hydrogen peroxide.

The invention relates to an electrochemical analysis method using redox-(re)cycling and to associated arrangements having an electrode system comprising at least three electrodes, with at least one working electrode, one counter electrode and one reference electrode being present. In addition, the invention also relates to specific uses of the arrangements having the electrode system.

In a specific form of amperometric analysis, the reduced form of a substance A_{red} which is to be detected is oxidized into its oxidized form Aₒₓ at a working electrode Wₒₓ and is reduced again to A_{red} at an adjacent working electrode W_{red}. This operation, which is known as redox-(re)cycling (literature: K. Aoki et. al., J. Electro-anal. Chem., 256 (1988), pages 269 to 282, O. Niwa et al., Anal. Chem., 65 (1993), pages 1559 to 1563), leads to signal amplification. A sensor arrangement with an electrically actuable array corresponding to WO 00/62048 A is particularly suitable for a method of this type. In addition to the working electrodes, there are in this case further auxiliary electrodes, but these are not able to record a redox potential.

Therefore, it is an object of the invention to provide a method and associated devices of the type described in the introduction in which the measurements in electrochemical analysis methods are improved and simplified and/or in which the measurement arrangement can be produced at lower cost.

According to the invention, in terms of the method the object is achieved by the measures described in patent claim 1, and in terms of the device the object is achieved by means of the features described in patent claim 5. Refinements to the invention are given in the dependent

method and/or device claims. Furthermore, the patent claims also give preferred uses of the arrangements with electrode system according to the invention which are described.

In the method according to the invention, redox-(re)cycling which is known per se is carried out in order to amplify evaluation signals for an electrochemical analysis method in which now, for the first time, an accurately defined redox potential is used for evaluation. Therefore, the invention in particular defines a reference electrode, at which the redox potential is tapped without current, i.e. with a high impedance, and is fed for further signal processing.

It is true that reference electrodes are already frequently used in electroanalytical methods (cf. for example W. Buchberger “Elektrochemische Analyseverfahren” [Electrochemical Analysis Methods] Spektrum Akademischer Verlag Heidelberg (1998), Berlin) which have to supply a stable reference-ground potential which is independent of the analyte. An example of the conventional reference electrode is the Ag/AgCl electrode which comprises the following arrangement:

El Conductor/silver/silver chloride/KCl solution/diaphragm. Reference electrodes of this type are of relatively complex structure and require volumes of a few cm³.

Nowadays, many electroanalytical methods are miniaturized with the aid of microelectronics and Microsystems technology (volume of a few mm³), but the extent to which reference electrodes can be miniaturized is limited. By way of example, it is possible to form an Ag/AgCl layer using thin-film technology and to add a defined KCl solution. A reference electrode of this type (referred to as an electrode of the 2nd Type) is used for a micro-multielectrode arrangement in DD 301 930 A9.

However, for microelectroanalytical methods, it is generally undesirable to use Ag/AgCl layers. Reasons for this include

an inevitable risk of contamination, an absence of process compatibility and high costs. To this extent, the reference electrodes which are known from the prior art are unsuitable for redox-(re)cycling, and consequently hitherto have not been used for this purpose.

When the invention is implemented, the associated arrangements include particular positioning of the reference electrode in the electrode system, specifically in such a way that all the measurement electrodes are in a symmetrical relationship with respect to the reference electrode. In this context, it is known per se and automatically assumed that what is known as a redox pair, i.e. a mixture of oxidized and reduced forms of a substance A, if appropriate a compound, in solution at an electrochemically active (precious) metal electrode forms what is known as a redox potential which is precisely defined. This redox potential can be measured without current by realizing the high-impedance reference electrode and can be utilized for evaluation.

In the present context, an electrochemically or catalytically active electrode is understood as meaning that both the oxidized form of the substance on the electrode material is chemisorbed, in order to allow exchange of electrons. This requirement is satisfied in particular by precious metals, such as gold (Au), platinum (Pt) or the like, but may also be satisfied by other materials, for example carbon, provided that they allow a redox reaction.

For a sensor based on the redox-(re)cycling method, it is necessary to have working electrodes W_{red} and Wₒₓ, a counter electrode C and a reference electrode R. One working electrode may be sufficient, if the counter electrode also acts as a working electrode. In the individual arrangements according to the invention, the reference electrode is configured and positioned in a particularly advantageous way with a view to potential measurement. It is preferable for all the electrodes to be formed from the same material, in particular a precious metal, such as for example gold.

This results in a crucial advantage in the fabrication of structures of this type using semiconductor technology. The use of conventional reference electrode materials, such as for example silver/silver chloride in accordance with DD 301 930 A, causes problems in semiconductor technology. Introducing silver/silver chloride into a semiconductor technology process would entail a high risk of contamination to the standard semiconductor processes and cause compatibility problems and high fabrication costs. The inventive design of the reference electrode using the same technology as that of the other electrodes, i.e. working electrodes and counter electrodes, avoids these problems and reduces the fabrication costs.

Further details and advantages of the invention will emerge from the following description of the figures illustrating exemplary embodiments with reference to the drawing in conjunction with the patent claims. In the drawing:

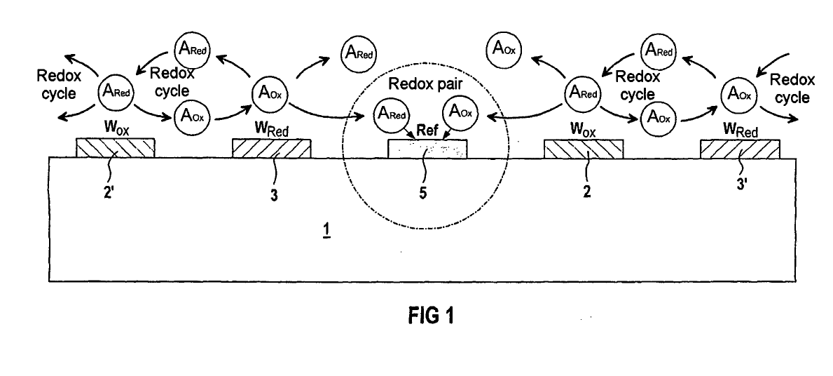

FIG. 1 diagrammatically depicts an arrangement for redox-(re)cycling, revealing in particular the positioning of the redox reference electrode according to the invention,

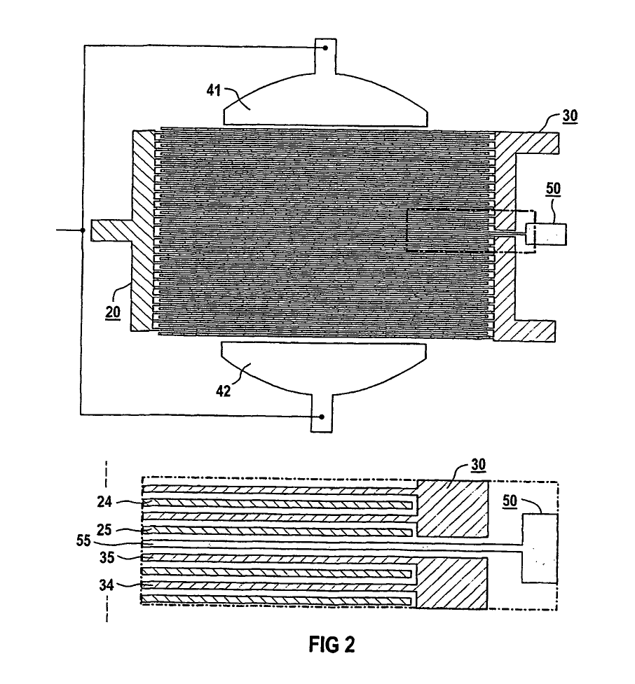

FIG. 2 diagrammatically depicts a plan view of an electrode system having two working electrodes, counter electrode and reference electrode for use in the arrangement shown in FIG. 1,

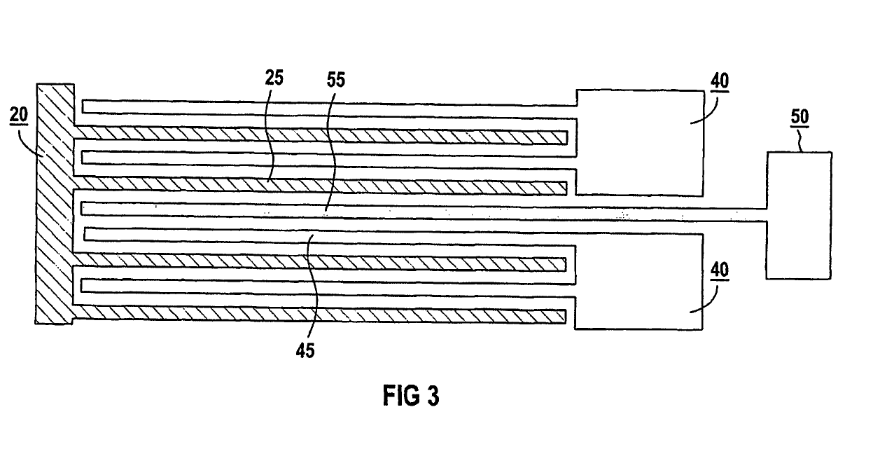

FIG. 3 diagrammatically depicts a modification to FIG. 2 with only one working electrode, counter electrode and reference electrode,

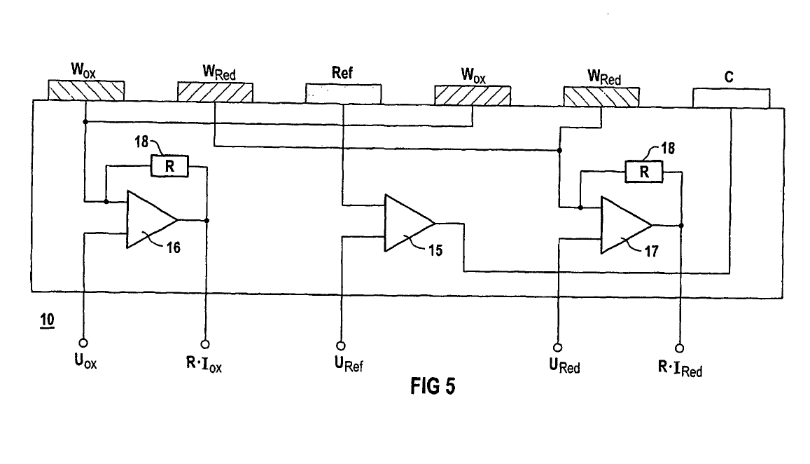

FIG. 5 diagrammatically depicts a cross section through a substrate with working electrodes and a reference electrode at the surface and a processing circuit in the interior of the semiconductor.

In an electrochemical analysis method, what is known as redox-(re)cycling is used. redox-(re)cycling is a known cyclic process for amplifying signals,

for which a structure as shown in FIG. 1 is used. There is a substance A, and it can be seen that the oxidized species A_{Ox}, are reduced at the working electrode W_{Red}, and that the reduced species A_{Red}, by contrast, are oxidized at the working electrode W_{Ox}. The working electrodes W_{Red} and W_{Ox} are also referred to as redox electrodes for what is known as a redox pair comprising the species A_{Red} and A_{Ox}.

In detail, FIG. 1 shows a section through part of an electrode arrangement having in each case an plurality of working electrodes 2, 2′ and 3, 3′ and a reference electrode 5, which are located on a substrate 1. The redox-(re)cycling, in the regions of the individual working electrodes 2, 2′ and 3, 3′, leads to the reactions indicated, so that a redox potential is formed. The reference electrode 5 is connected to the high-impedance input of a measurement amplifier (not shown in FIG. 1), with the result that the species A_{Ox} and A_{Red}, which diffuse onto the reference electrode 5 from both sides, form a redox potential which, in accordance with the Nernst equation, turns out as:

E=E₀+RT/zF*ln(C(A_{Ox})/C(A_{Red})) (1)

in which

E: Denotes the redox potential

R: Denotes a gas constant

T: Denotes the absolute temperature

z: Denotes the number of redox electrons

F: Denotes the Faraday constant

C: Denotes the concentration of the species (A_{Ox}) and (A_{Red})

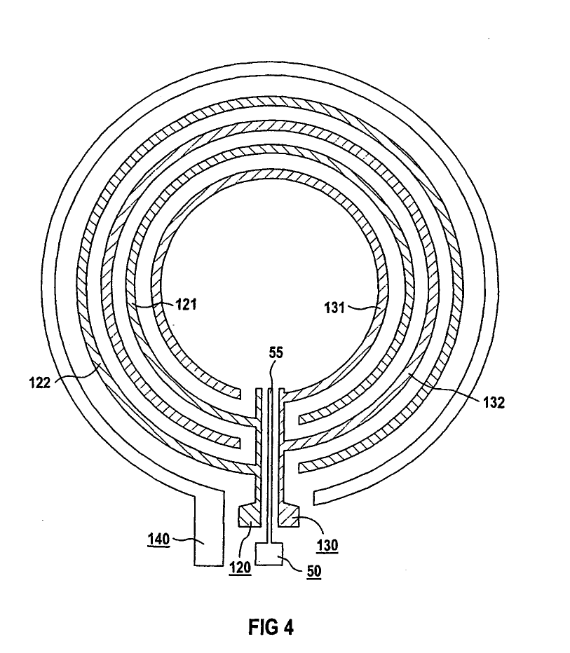

FIG. 4 diagrammatically depicts a plan view of an electrode system with electrode fingers formed parallel in the shape of a circle, and measuring technology for use as reference potential in redox-(re)cycling requires a high-impedance reference electrode. Another crucial factor in the function of a reference electrode principle of this type is that the redox potential is not dependent on the

The recording and evaluation of a redox potential of this nature by means of suitable electrochemical absolute concentrations of the oxidized and reduced species, but rather on their activities, i.e. chemical concentration ratio C(A_{Ox})/C(A_{Red}). In an ongoing redox-(re)cycling process which is in equilibrium, this concentration ratio A(A_{Ox})/C(A_{Red}) is equal to 1.

An example of a redox pair which can be cited is p-aminophenol/quinoneimine:

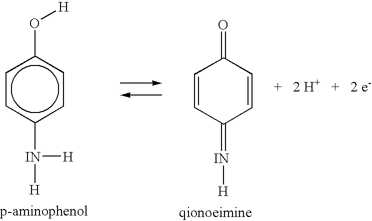

2 electrons and 2H⁺ ions are involved in the corresponding redox process.

This system is used, for example, for enzyme-linked detection reactions. In this case, the enzyme used as labelling or amplifying substance is “alkaline phosphatase”. Alkaline phosphatase is able to split p-aminophenyl phosphate into p-aminophenol and phosphate:

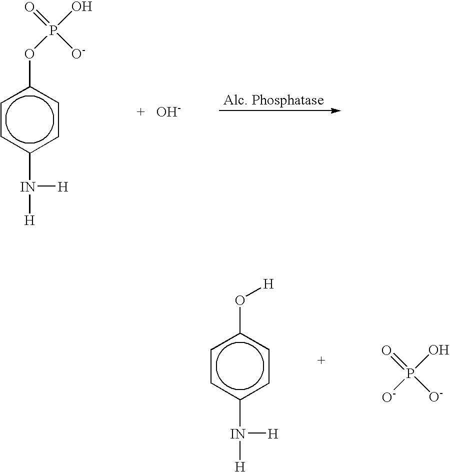

p-aminophenyl phosphate

The p-aminophenol which forms is oxidized at the electrode system or the p-aminophenol/quinoneimine redox pair is cyclized.

current, variations of this order of magnitude (60 mV) have a negligible influence. The redox-(re)cycling will at least commence, with the result that the ratio of p-aminophenol to quinoneimine moves closer to 1 and therefore the deviations in the reference electrode voltage also tend to zero.

At the start of a detection reaction of this type which is based on redox-(re)cycling, in theory there is not yet any quinoneimine, but rather only small quantities of p-aminophenol, with the result that the concentration ratio of the p-aminophenol/quinoneimine redox pair would differ significantly from 1 and according to the Nernst equation would lead to a shift in the redox potential.

However, the fact that the enzyme substrate p-aminophenyl phosphate is always in partially hydrolyzed form and therefore there are traces (approx. 0.1%) of p-aminophenol, which in turn are partially oxidized, so that there are small concentrations of the p-aminophenol/quinoneimine redox pair, is advantageous for the function of the reference electrode.

On account of the logarithmic relationship in Equation (1), there are only relatively minor deviations in the redox potential even if there are considerable differences in the concentration of oxidized and reduced form.

The latter can be explained by means of the following example: assuming that the p-aminophenol/quinoneimine ratio were not 1 but rather 100/1, i.e. only 1% of the p-aminophenol had been oxidized to form quinoneimine, the result, on the basis of the Nernst equation, would be a difference in the redox potential and therefore in the reference electrode voltage of only approx. 60 mV. Therefore, a deviation of 60 mV would result for the two working electrodes. The redox recycling operation is scarcely disturbed by this deviation, since the voltage difference between the two working electrodes (W_{Ox} and W_{Red}) is approx. 400 mV, and on account of the operation of the electrodes in the limiting diffusion

The use of the redox reference electrode according to the invention is not restricted to the particular situation of the p-aminophenol/quinoneimine system. It can be used for all enzyme-linked redox-(re)cycling processes. To stabilize the redox reference electrode potential, a small, defined quantity of the redox pair involved or any desired redox pair can be added to the enzyme substrate. A small quantity of the redox pair would, by means of its concentration ratio (=1), define the redox potential without distorting the analytical detection, since the analytical information is obtained from the enzyme-linked, rate of rise in the concentrations and is not dependent on the starting concentration.

The redox reference electrode according to the invention can also be applied to other analysis methods which are not enzyme-linked. A first example is the measurement of the oxygen partial pressure pO₂. A known method is based on the reduction of oxygen (O₂) and reaction to form OH⁻ at a catalytically active precious metal cathode in accordance with Equation (2):

½O₂+H₂O+2 e⁻—→2OH⁻ (2)

If an oxidizing potential which is sufficient to oxidize the OH⁻ formed back to O₂ is imposed on an anode designed as a second working electrode, a redox-(re)cycling process is set in motion. As an alternative to a second working electrode, it is also possible to use the counter electrode, at which the required potential is established automatically by means of the potentiostat. In this case too, the reference potential required for evaluation can be tapped using a redox reference electrode.

A further example is a glucose measurement. For this purpose, glucose is reacted with oxygen (O₂) to form gluconic acid, and hydrogen peroxide (H₂O₂) is generated by means of a cyclized process,

it also being possible to measure a reference potential for evaluation, by means of redox-(re)cycling.

FIG. 2 shows an embodiment for a four-electrode system, comprising the two working electrodes W_{Ox} and W_{Red}, which in this case are specifically designed as what are known as interdigitated electrodes 20 and 30, a counter electrode C, which for reasons of symmetry is designed with two electrically connected part-electrodes 41, 42, and a reference electrode R.

In the present context, an “interdigitated electrode” is understood as meaning an electrode with finger-like electrode parts, it being possible for two interdigitated electrodes to engage in one another in comb-like fashion by means of the corresponding fingers. This means that the working electrode 20 includes parallel fingers 21, 22, . . . , 25, . . . and the working electrode 30 includes parallel fingers 31, 32, . . . , 35, . . . .

The reference electrode 50 is designed as a “finger electrode” having a single finger 55 and is arranged in the double comb structure of the working electrodes 20 and 30 in such a manner that the finger 55 adjoins both a “finger” 25 as part of the working electrode 20 and a “finger” 35 as a partial region of the working electrode 30. The detailed excerpt presented in FIG. 2 also reveals a constant spacing between the reference electrode finger 55 and these partial regions of the working electrodes 20 and 30. Consequently, the conditions are precisely symmetrical.

As explained above, the arrangement shown in FIG. 2 comprises four electrodes, i.e. two working electrodes W_{Ox} and W_{Red}, a reference electrode R positioned in accordance with the invention and one or two counter electrodes C which, however, are in electrical contact with one another and to this extent represent a single electrode. An arrangement of this type with two working electrodes has been tried and tested in practice for recording redox potentials.

In FIG. 3, the arrangement shown in FIG. 2 has been modified to the effect that the counter electrode is used instead of the second working electrode 30 shown in FIG. 2. What this means is that, in addition to the working electrode W, which is designed in the same way as the working electrode 20 with fingers 21, 22, . . . , 25, . . . , the counter electrode 40 is identical in form, with individual fingers 41, 42, . . . , 45, . . . , the working electrode W and the counter electrode C in this case engaging in one another in comb-like fashion by means of their fingers 21, 22, 25, . . . and 41, 42, 45, . . . The finger 55 of the reference electrode 50 is at precisely the same distance from the two fingers 25 and 45 of the electrodes 20 and 40.

FIG. 4 illustrates an electrode arrangement which in terms of its basic structure is already known from DE 196 10 115 A1. The electrode arrangement shown in FIG. 4 likewise has two working electrodes W_{Ox} and W_{Red} with individual fingers, which in this case are formed parallel in the shape of a circle. In detail, this means that, starting from two parallel and radial electrode connections 120 and 130, individual fingers 121, 122, . . . and individual fingers 131, 132, . . . each run coaxially parallel but in opposite directions, so that overall they cover a circular area. An electrode arrangement of this type is compact in terms of area. An annular counter electrode 140 is arranged around the circular structure.

Furthermore, in FIG. 4 there is also a reference electrode 50 with a single finger 55, which runs parallel between the connections 120 and 130 of the working electrode W_{Ox} and W_{Red}, radially with respect to the overall system. The finger 55 of the reference electrode is therefore equally adjacent to partial regions of the working electrodes W_{Red} and W_{Ox} and at equal distances from these parts. This means in particular that the finger of the reference electrode does not project into the center of the arrangement.

FIG. 5 illustrates a structure substantially corresponding to that shown in FIG. 1, once again with a plurality of working electrodes W_{Ox} and W_{Red}, and associated reference electrode R and a counter electrode C on a substrate, which is denoted in this figure by 10. The substrate 10 used is, for example, silicon which is crystallographically oriented and also carries electric circuit elements for the metrological evaluation and/or amplification of the redox potential. The figure illustrates amplifiers 15 to 17 and feedback resistors 18, the high-impedance operational amplifier 15 being of particular importance to the operation of the reference electrode R in order to allow current-free measurement of the redox potential. The reference electrode R is at the high-impedance input of the amplifier 15, its output being connected to the counter electrode C. The desired reference potential U_{Ref} is preselected via the other input of the amplifier 15.

The voltage drop U=R·Iₒₓ can be measured by means of the further operational amplifiers 16 and 17 given connection to the working electrode Wₒₓ and the associated potential Uₒₓ, and the variable Iₒₓ can be determined if the resistance R of the feedback resistor 18 is known. The same is true of I_{Red} at the working electrode W_{red}.

To realize the arrangement shown in FIG. 3, it is merely necessary to polarize one working electrode interdigitated electrode and to measure the resulting electric current. The second interdigitated electrode is connected as a counter electrode, i.e. the polarization of this electrode is set automatically by means of the output of the potentiostat.

The arrangement shown in FIG. 5 can be broadened in a simple way to form a one-dimensional or two-dimensional array. As an alternative to silicon, it is also possible to use other materials, for example plastic, glass or ceramic, for the substrate. In this

case, the evaluation circuit is composed of discrete components.

In FIG. 5, it is pertinent that the reference electrode is directly incorporated in an evaluation circuit, which may be of either analog or digital structure. The direct linking of the reference electrode to the high-impedance input of the amplifier allows substantially interference-free measurement of the redox potential by means of electrochemical measurement signals which are present.

This is particularly important because what are known as the exchange current densities of the redox pair are very low at the “ultramicro” reference electrode, i.e. this reference electrode has a very high impedance and a very low capacitance. Therefore, the input of the amplifier has to have a very high impedance and a very low input capacitance. This requirement is satisfied particularly well, for example, by an MOS transistor positioned directly below the electrode.

Overall, the electrode system together with the associated substrate forms a complete measuring arrangement for electrochemical analysis methods, which is suitable not only for the redox-(re)cycling which has been described in detail with reference to a specific example, but also for recording enzyme-linked detection reactions.

1. An electrochemical analysis method by means of redox-(re)cycling, comprising the following method steps:
the reduced form of a substance is oxidized at an electrode, and the oxidized form of the substance produced is reduced to the original form of the substrate at another electrode, so that together what is known as a redox pair is formed,
signal amplification for subsequent signal evaluation is effected by a cyclic sequence of oxidation and reduction at the two electrodes, known as the redox electrodes,
a redox potential which is dependent on the ratio of the concentrations or activities of the redox pair and forms at a catalytically active surface is tapped without current and, as reference-ground potential, is used as the basis for the signal evaluation by means of electrochemical measurement technology.

2. The electrochemical analysis method as claimed in claim 1, characterized in that the redox potential is used as reference potential in the signal amplification by the redox-(re)cycling.

3. The electrochemical analysis method as claimed in claim 2, characterized in that the redox potential is tapped at a separate reference electrode, which is connected to a high-impedance amplifier.

4. The electrochemical analysis method as claimed in claim 3, characterized in that, to tap an exact and stable redox potential, the reference electrode is positioned equidistantly with respect to the redox electrodes.

5. An arrangement for an electrochemical analysis method for carrying out the method as claimed in claim 1 or one of
claims 2 to 4, having an electrode system comprising at least three electrodes, with at least one working electrode, one counter electrode and one reference electrode being present, characterized in that the reference electrode (R) is adjacent to at least partial regions of two of the other electrodes (Wₒₓ, W_{red}, C).

6. The arrangement as claimed in claim 5, characterized in that the reference electrode (R) is at an equal distance from the adjacent partial regions of the other electrodes (Wₒₓ, W_{red}, C).

7. The arrangement as claimed in claim 5, characterized in that one working electrode (W), one counter electrode (C) and one reference electrode (R) are present.

8. The arrangement as claimed in claim 5, characterized in that two working electrodes (Wₒₓ, W_{red}), one counter electrode (C) and one reference electrode (R) are present.

9. The arrangement as claimed in claim 5 and claim 8, with two working electrodes present, characterized in that the working electrodes (Wₒₓ, W_{red}) are of identical design, each being in the form of a comb with individual fingers (21, 22, . . . , 25, . . . ), and in that the working electrodes (Wₒₓ, W_{red}) engage in one another by means of the individual fingers (31, 32, . . . , 35, . . . ), the reference electrode (R) being adjacent both to a finger (25) of the first working electrode (Wₒₓ) and a finger (35) of the second working electrode (W_{red}).

10. The arrangement as claimed in claim 5 and claim 7, with one working electrode present, characterized in that the working electrode (W) and the counter electrode (C) are of identical design and are each in the form of a comb with
individual fingers, and in that the individual fingers (21, 22, . . . , 25, . . . 41, 42, . . . , 45, . . . ) of the working electrode (W) and the counter electrode (C) engage in one another, the reference electrode (R) being adjacent to both a finger (20) of the working electrode (W) and a finger (45) of the counter electrode (C).

11. The arrangement as claimed in claim 8 and claim 9, characterized in that the working electrodes (W_{Ox}, W_{red}) and the reference electrode (R) run parallel and form a rectangular area.

12. The arrangement as claimed in claim 7 and claim 10, characterized in that the working electrode (W), the counter electrode (C) and the reference electrode (R) run parallel and form a rectangular area.

13. The arrangement as claimed in claim 8 and claim 9, characterized in that the working electrodes (Wₒₓ, W_{red}) and the reference electrode (R) run parallel in the shape of a circle and form a circular area.

14. The arrangement as claimed in claim 13, characterized in that the working electrodes (Wₒₓ, W_{red}) and the counter electrode (C), starting from radial connections (120, 130), run parallel in the shape of a circle and form the circular area, the reference electrode (R) running radially and parallel between the connections (120, 130) of the working electrodes (Wₒₓ, W_{red}).

15. The arrangement as claimed in one of claims 5 to 14, characterized in that all the electrodes (Wₒₓ, W_{red}, C, R) consist of the same material.

16. The arrangement as claimed in claim 15, characterized in that the electrodes (Wₒₓ, W_{red}, C, R) are formed from precious metal.

17. The arrangement as claimed in claim 15, characterized in that the electrode system is arranged on a planar substrate (1) made from a suitable material, such as for example plastic, glass, ceramic or in particular silicon.

18. The arrangement as claimed in claim 15, characterized in that the substrate is a crystallographically oriented silicon substrate (10).

19. The arrangement as claimed in claim 18, characterized in that the reference electrode (R) is connected to the high-impedance input of a measurement amplifier (15), the measurement amplifier (15) being formed by integration in the silicon substrate (10) and allowing interference-free potential measurement on account of the short connection distance.

20. The arrangement as claimed in one of claims 5 to 19, characterized in that the silicon substrate (10) together with the electrodes (Wₒₓ, W_{red}, C, R) forms an array system.

21. The use of the arrangement having at least one electrode system as claimed in claim 5 or one of claims 6 to 20 for measuring enzyme-linked detection reaction.

22. The use of the arrangement having at least one electrode system as claimed in claim 5 or one of claims 6 to 20 for measuring an oxygen partial pressure (pO₂).

23. The use of the arrangement having at least one electrode system as claimed in claim 5 or one of claims 6 to 20 for measuring hydrogen peroxide (H₂O₂) for glucose determination.
